(19) United States
(12) Patent Application Publication
(10) Pub. No.: US 2024/0139431 A1
(43) Pub. Date: May 2, 2024

(54) AUTO-INJECTOR PLUNGER DRIVER LATCH

(71) Applicant: Owen Mumford Limited, Woodstock, Oxfordshire (GB)

(72) Inventor: Matthew David FARMER, Woodstock, Oxfordshire (GB)

(21) Appl. No.: 18/547,578

(22) PCT Filed: Feb. 6, 2022
(86) PCT No.: PCT/EP2022/052799

§ 371 Date: Aug. 23, 2023

(30) Foreign Application Priority Data
Feb. 28, 2021 (GB) 2102847.7

(51) Int. Cl. A61M 5/32; A61M 5/00; A61M 5/20

(52) U.S. Cl. CPC A61M 5/326; A61M 5/002; A61M 5/2033; A61M 2005/2013

(57) ABSTRACT

An auto-injector comprising: a housing for receiving a syringe having a barrel containing a medicament, a plunger and a needle; a plunger driver configured to act on the syringe plunger to move the syringe plunger through the housing during a needle insertion phase and an injection phase; a safety shroud; and a plunger driver latch coupled to said safety shroud and moveable between first and second positions and configured to: while in the first position before the needle insertion phase, latch the plunger driver at a pre-insertion position in the housing, move to the second position as a result of pressure applied to the safety shroud, to unlatch the plunger driver from the pre-insertion position and allow forward movement of the plunger driver to a post-insertion and injection position in the housing and latch the plunger driver at the post-insertion and injection position, and return to the first position upon removal or reduction of pressure on the safety shroud, to unlatch the plunger driver from the post-insertion and injection position and allow forward movement of the plunger driver to deploy the safety shroud.

### TECHNICAL FIELD

The invention relates to auto-injectors for use with syringes. The invention may relate to, but need not be limited to, auto-injectors for use with syringes having a safety shroud.

### BACKGROUND

Auto-injectors are devices for receiving and operating syringes automatically on activation by a user. Auto-injectors typically include a plunger driver that engages with a plunger of a syringe inserted into the auto-injector and a drive spring that are arranged to couple to and apply a force to the plunger of the syringe inserted into the auto-injector. To inject a drug, the user holds the auto-injector against the injection site and triggers the auto-injector, causing the plunger driver to drive the plunger of the syringe forward into a drug-filled barrel of the syringe. The drug is thereby forced through a needle into the injection site.

Some auto-injectors include a safety shroud that is deployed during or after the injection is complete. The safety shroud covers the needle as the auto-injector is removed from the injection site to prevent accidental needle stabbing during post-injection handling of the auto-injector. However, if the auto-injector is of a reusable type, the user still needs to remove the syringe from the auto-injector thereby exposing the needle and increasing the risk of injury. To minimise this risk, the syringe itself instead of the auto-injector may be provided with a safety shroud. In such cases, the auto-injector is provided with a mechanism to deploy the safety shroud of the syringe.

Syringes with their own safety shroud may be broadly split into ‘active’ and ‘passive’ syringes. Active syringes with a safety shroud typically require some action by a user to deploy the safety shroud, for example the pressing of a trigger button. Such action may be taken during or after removal of the needle from the injection site. Passive syringes with a safety shroud on the other hand deploy the safety shroud without any specific action by the user beyond that which is usually taken to use the syringe. For example, movement of a plunger beyond a certain point during injection of a drug may trigger the release of a spring-loaded safety shroud or, in the case of an auto-injector, the auto-injector plunger driver may bias the safety shroud towards its closed position. When the safety shroud is biased towards the closed, locked out position in this way, there is a risk of premature deployment and lock out of the safety shroud, for example because the spring force of the drive spring deploying the shroud is enough to force the auto-injector off the injection site thereby pushing the shroud to its deployed position.

An exemplary auto-injector is proposed in WO/2020/064927A1.

### SUMMARY

According to a first aspect, there is provided an auto-injector comprising: a housing for receiving a syringe having a barrel containing a medicament, a plunger and a needle; a plunger driver configured to act on the syringe plunger to move the syringe plunger through the housing during a needle insertion phase and an injection phase; a safety shroud; and a plunger driver latch coupled to said safety shroud and moveable between first and second positions. The plunger driver latch is configured to: while in the first position before the needle insertion phase, latch the plunger driver at a pre-insertion position in the housing, move to the second position as a result of pressure applied to the safety shroud, to unlatch the plunger driver from the pre-insertion position and allow forward movement of the plunger driver to a post-insertion and injection position in the housing and latch the plunger driver at the post-insertion and injection position, and return to the first position upon removal or reduction of pressure on the safety shroud, to unlatch the plunger driver from the post-insertion and injection position and allow forward movement of the plunger driver to deploy the safety shroud.

Optionally, before deployment of the safety shroud, the safety shroud is moveable between an extended position and a retracted position in the housing as a result of said applying, or removal or reduction of pressure.

Optionally, before deployment of the safety shroud, movement of the safety shroud from the extended position to the retracted position moves the plunger driver latch from the first position to the second position to unlatch the plunger driver from the pre-insertion position.

Optionally, movement of the safety shroud from the retracted position to the extended position returns the plunger driver latch to the first position to unlatch the plunger driver from the post-insertion and injection position.

Optionally, the safety shroud comprises a first angled cam surface configured to engage a second angled cam surface of the plunger driver latch to move the plunger driver latch to the second position when the safety shroud is moved to the retracted position.

Optionally, the plunger driver latch comprises a pivot; and wherein the plunger driver latch is rotatable about the pivot to move between the first position and the second position.

Optionally, the plunger driver latch is configured to: rotate about the pivot from the first position to the second position responsive to said engagement between the first angled cam surface and the second angled cam surface, and rotate about the pivot from the second position to the first position responsive to disengagement of the first angled cam surface from the second angled cam surface.

Optionally, the auto-injector comprises a biasing member configured to bias the plunger driver latch towards the first position.

Optionally, wherein the biasing member comprises a spring.

Optionally, the plunger driver comprises an engagement member; wherein the plunger driver latch comprises a first blocking surface and a second blocking surface; wherein when the plunger driver latch is in the first position, the first blocking surface of is configured to engage the engagement member of the plunger driver to latch the plunger driver at the pre-insertion position; and wherein when the plunger driver latch is in the second position, the second blocking surface is configured to engage the engagement member of the plunger driver to latch the plunger driver at the post-insertion and injection position.

Optionally, the plunger driver is configured to decouple from the plunger of the syringe at the post-insertion and injection position.

Optionally, after said unlatching of the plunger driver from the post-insertion and injection position, the plunger driver is configured apply a continuous forward force to the safety shroud to deploy the safety shroud.

Optionally, the plunger driver is configured to lock out the safety shroud on deployment thereof.

Optionally, the auto-injector comprises an openable lid to allow insertion and removal of the syringe, wherein the lid is configured to hinge between an open position and a closed position.

Optionally, the lid is configured on a closing movement thereof to prime a drive spring of the plunger driver.

According to a second aspect, there is provided an auto-injector according the above aspect in combination with a syringe comprising a syringe safety shroud configured to cover a needle of the syringe when in a closed position.

Optionally, after said unlatching of the plunger driver from the post-insertion and injection position, the plunger driver is configured apply a continuous forward force to the syringe safety shroud to deploy the syringe safety shroud.

Optionally, the plunger driver is configured to lock out the syringe safety shroud on deployment thereof.

According to a third aspect, there is provided a kit of parts comprising: the auto-injector of any of the above aspects; and a syringe comprising a syringe safety shroud configured to cover a needle of the syringe when in a deployed position.

Optionally, after said unlatching of the plunger driver from the post-insertion and injection position, the plunger driver is configured apply a continuous forward force to the syringe safety shroud to deploy the syringe safety shroud.

Optionally, the plunger driver is configured to lock out the syringe safety shroud on deployment thereof.

### BRIEF DESCRIPTION OF THE DRAWINGS

Exemplary embodiments are disclosed herein with reference to the accompanying drawings, in which:

FIG. 1a shows a view of an auto-injector.

FIG. 1b shows a view of the auto-injector of FIG. 1b with a safety syringe received therein.

FIG. 1c shows a partially cut away view of the auto-injector of FIG. 1a with a closed lid.

FIG. 2a shows a cross-sectional view of part of the auto-injector of FIGS. 1a-1b at a first stage of an injection.

FIG. 2b shows a cross-sectional view of the part of the auto-injector of FIG. 2a at a second stage of the injection.

FIG. 2c shows a cross-sectional view of the part of the auto-injector of FIG. 2a at a third stage of the injection.

FIG. 2d shows a cross-sectional view of the part of the auto-injector of FIG. 2a at a safety shroud deployment stage.

### DETAILED DESCRIPTION

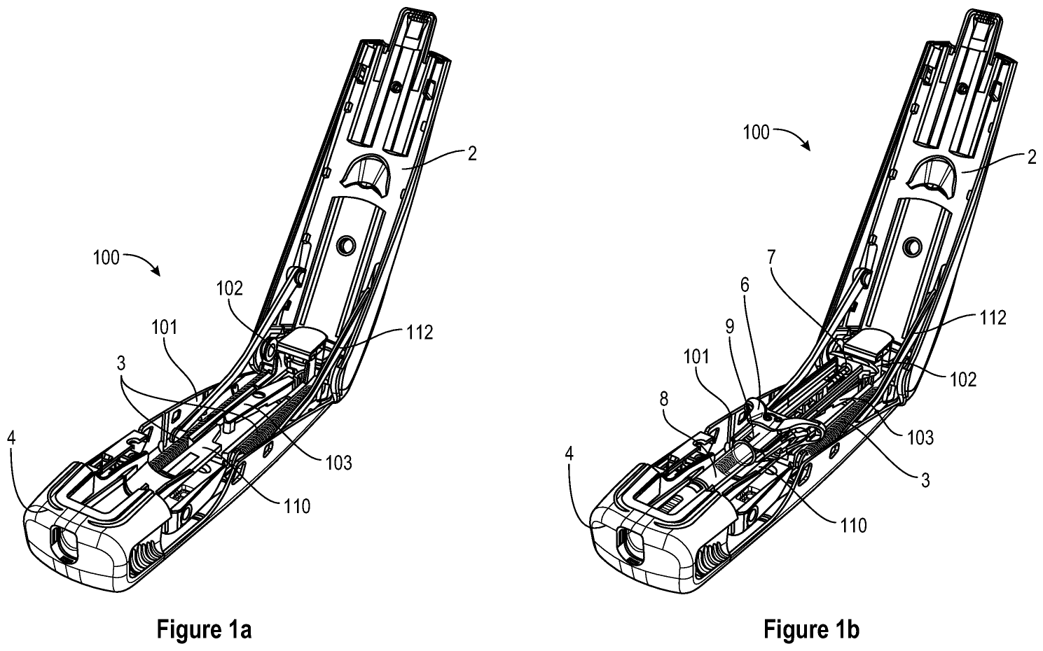

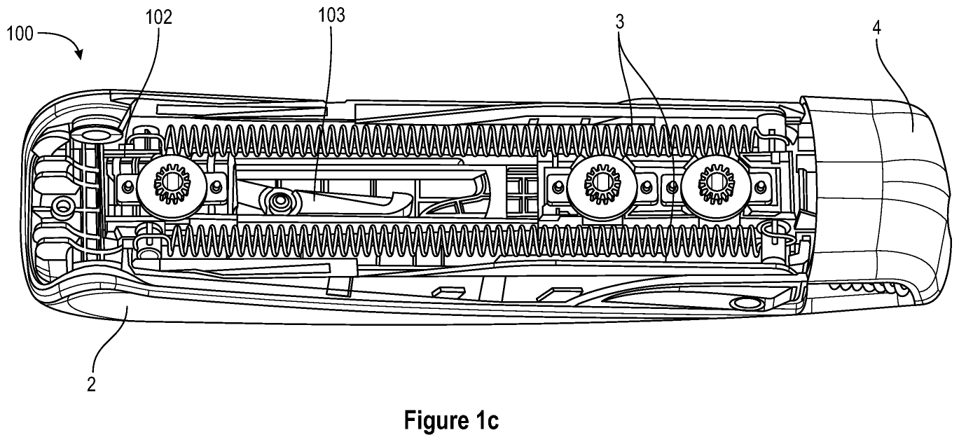

FIG. 1a shows a view of an auto-injector 100. The auto-injector comprises a housing 101 having a lid 2, a plunger driver 102 comprising one or more drive springs 3, a removable cap 4, a plunger driver latch 103 moveable between first and second positions and coupled to a safety shroud 110 of the auto-injector. The housing is configured for receiving a syringe having a barrel containing a medicament, a plunger and a needle. The lid 2 is configured to open to allow a user to position the syringe, for example a safety syringe, in the auto-injector 100 prior to use and to remove the syringe after use. The plunger driver 102 is configured to couple to a plunger of the syringe inserted in the auto-injector 100 for example by engaging a head of the plunger at its rear surface. Thus, the plunger driver is configured to act on the syringe plunger to move the syringe plunger through the housing during a needle insertion phase and an injection phase. The removable cap 4 covers a forward end of the auto-injector 100 prior to use. The safety shroud 110 is configured to move forwards and rearwards in the auto-injector 100, for example responsive to a user pressing and releasing a forward portion of the safety shroud 110 extending from a forward end of the auto-injector 100 onto an injection site (not shown).

FIG. 1b shows a view of the auto-injector of FIG. 1b with a safety syringe 6 received therein. The safety syringe 6 comprises a plunger 7 configured to, responsive to a force applied thereon by the plunger driver 102, move forwards within the auto-injector to force the contents of a barrel 8 of the safety syringe 6 through a needle (not shown) of the safety syringe 6 into an injection site. The safety syringe 6 further comprises a syringe safety shroud 9 configured to cover a needle of the safety syringe 6 at the end of an injection to allow for safe handling of the safety syringe 6 after the injection is complete and the safety syringe 6 is removed from the auto-injector. The syringe safety shroud 9 is configured to travel forwards over the barrel 8 coupled with the forward movement of the plunger 7, to decouple from the plunger 7 when the plunger reaches the end of the barrel 8 and continue further forward movement to cover a needle of the safety syringe 6 inside the housing 100 after the injection.

FIG. 1c shows a partially cut away view of an underside of the auto-injector 100 of FIG. 1a with a closed lid 2. The safety syringe is not shown in FIG. 1c. The drive springs 3 of the plunger driver 102 are primed and the plunger driver 102 is prevented from being driven forward by the primed drive springs 3 by a plunger driver latch 103. The plunger driver latch 103 is positioned under or adjacent the path that the plunger driver 102 and/or syringe plunger 7 take when driving a plunger of the syringe during an injection.

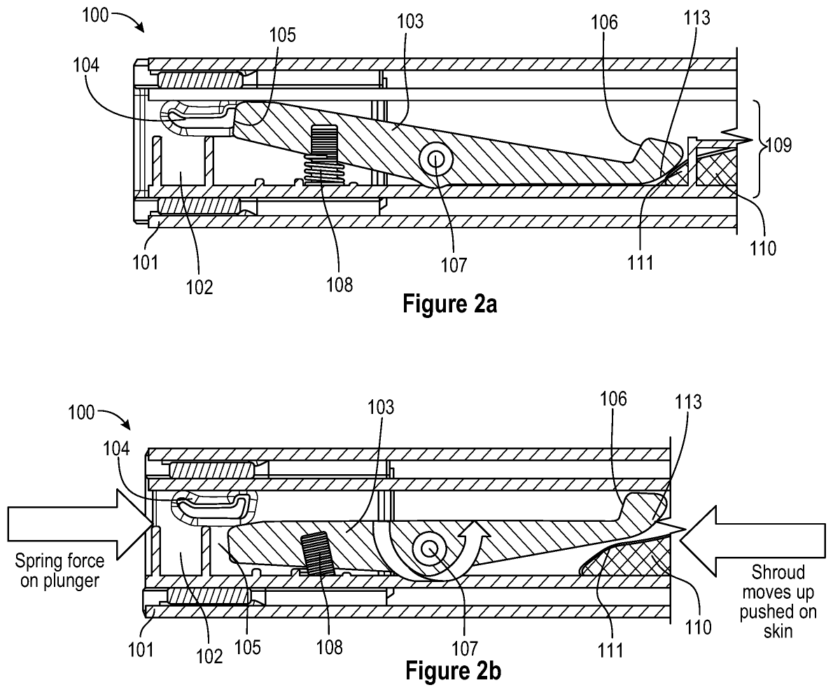

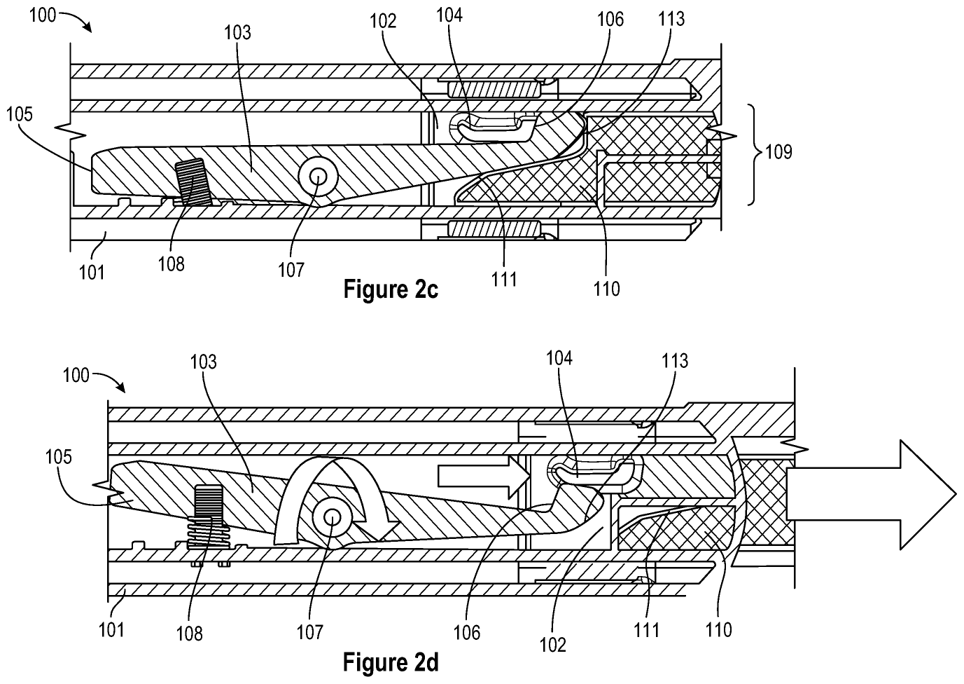

Further details of the plunger driver 102 and the plunger driver latch 103 will now be described with reference to FIGS. 2a-2d.

FIG. 2a shows a cross-sectional view of part of the auto-injector 100 of FIGS. 1a and 1b at a first stage of an injection after the removable cap has been removed from the auto-injector 100. As described above, the auto-injector 100 comprises a housing 101 for receiving a syringe, a plunger driver 102 that may comprise one or more drive springs, and a plunger driver latch 103 moveable between a first and a second position and coupled to the safety shroud 110. When the plunger driver latch 103 is in the first position before the needle insertion phase as shown in FIG. 2a, the plunger driver latch 103 latches the plunger driver at a pre-insertion position in the housing. To achieve this, the plunger driver 102 comprises an engagement member 104 configured to engage with a first blocking surface 105 of the plunger driver latch 103 when the plunger driver latch 103 is in a first position. With the plunger driver latch 103 positioned alongside the path of movement of the plunger driver 102 and the syringe plunger, the engagement member 104 protrudes sideways from the plunger driver 102 to enable the engagement with the blocking surface 105 of the plunger driver latch 103.

The plunger driver latch 103 further comprises a second blocking surface 106 positioned at an opposite end of the plunger driver latch 103 to the first blocking surface 105. When the plunger driver latch 103 is in the first position, as in FIG. 2a, the second blocking surface 106 is positioned outside of the movement path the engagement member 104. Thus, when the plunger driver 102 is in the first position, the first blocking surface 105 latches the plunger driver 102 in place at a first point on the forward stroke of the injection, for example the at the beginning of the stroke before the needle insertion phase, while the second blocking surface 106 remains outside of the path of movement of the engagement member 104.

The plunger driver latch 103 further comprises a pivot 107 mounted on a support structure in the auto-injector 100 and the auto-injector 100 further comprises a biasing member 108 such as a spring configured to bias the plunger driver latch 103 towards the first position by applying a torque about the pivot 107. Biasing of the plunger driver latch 103 towards the first position ensures the engagement member 104 of the plunger driver remains engaged with the first blocking surface 105 of the plunger driver latch and thereby blocks movement of the plunger driver 102 from the pre-insertion position in the housing 101. The plunger driver latch 103 may be provided with an recess into which the biasing member 108 may be securely received when biasing the plunger driver latch 103 towards the first position.

Before the deployment of the safety shroud 110, the safety shroud 110 is moveable forwards and rearwards between an extended position and a retracted position in the housing, for example in a channel 109 of the housing. As described above, this forward and rearward movement of the safety shroud between the extended and retracted positions may be a result of the application, removal or reduction of pressure on the safety shroud, for example when a user presses and releases a forward portion of the safety shroud 110 extending from a forward end of the auto-injector housing onto an injection site (not shown). Due to the coupling between the plunger driver latch 103 and the safety shroud 110, the movement of the safety shroud 110 from the extended position to the retracted position moves the plunger driver latch 103 from the first position to the second position to unlatch the plunger driver 102 from the pre-insertion position. The coupling between the plunger driver latch 103 and the safety shroud 110 may comprise a first cam surface 111 on the safety shroud 110 and a second cam surface 113 on the plunger driver latch 103. Engagement between the first cam surface 111 and the second cam surface 113 as the safety shroud 110 is moved from the extended position to the retracted position moves the plunger driver latch 103 from the first position to the second position.

For example, during use of the auto-injector 100, the user presses the forward portion of the safety shroud 110 onto the injection site causing the safety shroud 110 to move rearwards in the channel 109 from the extended position to the retracted position. When the first angled cam surface 111 of the safety shroud 110 engages the second angled cam surface 113 of the plunger driver latch 103, the continued linear rearward movement of safety shroud 110 in the channel against the bias of the biasing member 108 of the plunger driver latch 103. is converted into a rotational movement of the plunger driver latch 103 about the pivot 107 forcing the plunger driver latch 103 into the second position. The plunger driver 102 is thereby unlatched from the pre-insertion position and is allowed to move forward under the force of the one or more driver springs.

FIG. 2b shows the autoinjector 100 of FIG. 2a with the plunger driver latch 103 in the second position.

As described above, as the plunger driver latch 103 moves into the second position, the first blocking surface 105 of the plunger driver latch 103 rotates out of the movement path of and disengages from the engagement member 104 of the plunger driver 102. At the same time, the second blocking surface 106, on the opposite side of the plunger driver latch 103 rotates into the movement path of the engagement member 104. Under the force of the one or more drive springs, the plunger driver 102 and the engagement member 104 move forward in the auto-injector thereby driving the plunger of the syringe into the barrel of the syringe (not shown) causing the contents of the barrel to be forced through a needle and into the injection site.

As the plunger driver 102 moves forward in the auto-injector, the engagement member 104, no longer blocked by the first blocking surface 105, travels along the plunger driver latch 103, beyond the pivot 107 and into engagement with the second blocking surface 106 at a second point on the forward stroke of the plunger driver 102 thereby latching the plunger driver 102 at a post-insertion and injection position. At the post-insertion and injection position, the one or more drive springs have not fully unloaded and are accordingly still under tension, compression and/or torsion depending of the spring used is a tension, compression and/or torsion spring.

The plunger driver latch 103 has a shape having a main body and a hooked-portion wherein the first blocking surface 105 is on an end of the main body, and the second blocking surface 106 is on the hooked-portion. Thus, as the plunger driver latch 103 rotates about the pivot 107, the main body moves out of the way of the engagement member 104 and the hooked portion is configured to catch the engagement member 104 at the post-insertion and injection position as the plunger driver 102 moves forward.

FIG. 2c shows the auto-injector 100 of FIG. 2a with the plunger driver latch 103 in the second position and the plunger driver 102 driver latched at the post-insertion and injection position.

As described above, responsive to a user pressing a forward portion of the safety shroud 110 onto an injection site, the safety shroud 110 has moved rearwardly into the channel 109 into the retracted position and the engagement of the first and second angled cam surfaces 111, 113 has rotated the plunger driver latch 103 about the pivot 107 against the bias of the biasing member 108. The engagement member 104 of the plunger driver 102 with the second blocking surface 106 of the plunger driver latch 103 blocks movement of the plunger driver 102 at the post-insertion and injection position on the forward stroke of the plunger driver 102.

By latching the plunger driver 102 at the post-insertion and injection position using the plunger driver latch 103, any remaining, unloaded forward force of the drive spring of the plunger driver 102 is applied to the plunger driver latch 103 rather than to the safety shroud 110. As the plunger driver latch 103 is blocked from rotating about the pivot 107 by the rear portion of the safety shroud 110, the forward force of the drive spring of the plunger driver 102 is unable to act directly forwardly on the safety shroud 110. The risk of premature deployment and lock out of the safety shroud 110 that would otherwise have resulted from the forward force of the drive spring on the safety shroud 110 is thereby reduced when the plunger driver 102 is latched at the post-insertion and injection position.

When the plunger driver 102 is latched post-insertion and injection position as in FIG. 2c, the syringe plunger being driven by the plunger driver 102 may already be at the end of the barrel of the syringe (not shown) and the contents of the barrel fully injected into the injection site. In some cases, an injection protocol may require that the auto-injector is held on the injection site for a predetermined time to allow pressure from the injection in the injection site to dissipate. The auto-injector 100 may accordingly optionally be provided with electronics and a means to visually or audibly indicate to a user how long the user must hold the auto-injector 100 in position on the injection site at the end of the injection and to provide a countdown to ensure the user holds the auto-injector on the injection site for required time. The visual and/or audible indicator may comprise one or more light emitting diodes and/or a speaker configured to emit a sound. This allows the injection pressure to dissipate in the injection site, minimising the risk of splashback and ejection of the drug from the injection site.

After the user has held the auto-injector 100 on the injection site with the safety shroud 110 pressed rearwardly into the channel 109 in the retracted position for the predetermined time to complete the injection, the user moves the forward portion of the safety shroud 110 off the injection site. This permits the first angled cam surface 111 of the safety shroud 110 to disengage from the second angled cam surface 113 of the plunger driver latch 103. With the plunger driver latch 103 no longer blocked from pivoting about the pivot 107, the bias provided by the biasing member 108 rotates the plunger driver latch 103 to return it from the second position to the first position. This unlatches the plunger driver 102 from the post-insertion and injection position and allows forward movement of the plunger driver 102 to deploy the safety shroud.

FIG. 2d shows the auto-injector 100 of FIG. 2a with the plunger driver latch 103 back in the first position.

As described above, responsive to the user moving the forward safety shroud 110 off the injection site, the angled cam surfaces 111, 113 have disengaged and the plunger driver latch 103 has returned from the second position to the first position. With the plunger driver latch 103 back in the first position, the second blocking surface 106 of the plunger driver latch 103 is disengaged from the engagement member 104 of the plunger driver 102.

At the point when the contents of the barrel have been fully injected into the injection site and the plunger of the syringe has reached the end of its forward injection stroke, the plunger driver 102 may decouple from the plunger of the syringe. This allows the plunger driver 102 to move forward separately from the plunger of the syringe beyond the post-insertion and injection position to deploy the safety shroud. For example, under the remaining force of the one or more drive springs, the now decoupled plunger driver 102 continues its forward movement beyond the post-insertion and injection position where it was previously driver latched by the plunger driver latch 103.

As the plunger driver 102 continues its forward movement, the engagement member 104 engages with the safety shroud 110, forcing the safety shroud 110 into a deployed position where it is locked out covering the needle of the syringe. This movement at the same time moves the syringe safety shroud of the safety syringe received in the auto-injector into a locked out position covering the needle of the syringe. With both the auto-injector safety shroud and the syringe safety shroud deployed and locked out. The lid of the auto-injector 100 may be opened and the syringe may then be safely removed from the auto-injector without risk of accidental injury to the user.

Latching the plunger driver 102 at both the pre-insertion position and the post-insertion and injection position as described above allows the spring force of the drive springs of the plunger driver to be used to safely deploy the safety sheath of both the auto-injector 100 and the safety syringe received therein without the risk of the accidental safety shroud deployment. This has the further advantage that no separate shroud deployment spring with a relatively lower force is required.

As used herein, the term “coupled” encompasses a situation where there is corresponding linear movement of two coupled features. Coupled features may move in the same direction. Conversely, the term “decoupled” encompasses a situation where there is independent movement between decoupled features. In exemplary apparatus, a feature that is decoupled from another feature may be configured to move while the other feature remains stationary.

As used herein, the term forward and rearward refer respectively to a needle end of an auto-injector that is pressed against the user's skin and the end opposite the needle end. Forward and rearward movement thus refer to movement towards the needle end or movement away from the needle end of the auto-injector. The terms forward stroke thus refers to the total forward movement of a component of the auto-injector from a starting point towards the needle end to an endpoint. For example, the forward stroke of the syringe plunger refers to the movement towards the needle end required to fully inject the contents of the syringe barrel into a user. The forward stroke of the plunger driver 102 thus refers to the movement towards the needle end required to move from a starting to an endpoint. The endpoints of the forward stroke of the syringe plunger and the plunger driver may or may not coincide.

The skilled person will envisage further embodiments of the invention without departing from the scope of the appended claims.

For example, the drive spring that drives the plunger driver may comprise a compression spring configured to push the plunger driver forward in the auto-injector or a tension spring configured to pull the plunger driver forward. Alternatively, both may be used together to increase the injection force of the auto-injector.

Further, the housing may be provided with a lid configured to open and close to allow insertion of a syringe in the auto-injector. In a non-limiting example, the housing may comprise a hinged lid configured to open to allow insertion and removal of the syringe from the housing before and after use. Alternatively, the syringe may be inserted into the auto-injector by removal of a rear portion of the auto-injector and insertion of the syringe through the created opening.

Further, the lid may be configured to prime the drive springs on an opening and/or closing movement thereof. This may be achieved by, for example, coupling the lid to the drive springs, for example through the plunger driver, with one or more linkages 112. Movement of the lid in the opening or closing direction compresses or tensions the drive springs through the linkages 112 to prime the drive springs. For example, the linkages may be slidably coupled to the lid at one end and to the plunge driver at the other. As the lid moves, the linkages 112 slide in a channel In the lid thereby converting the movement of the lid as it hinges open or closed into a linear movement in the drive spring thereby compressing it or tensioning it depending on if the drive spring is a compression or a tension spring.

Further, the drive spring may be configured to be primed in two priming movements whereby an opening movement of the housing partially primes the drive spring and a closing movement of the housing completes the priming. This spreads the priming force required over two movements, each easier than a single priming movement.

1. An auto-injector comprising:
a housing for receiving a syringe having a barrel containing a medicament, a plunger and a needle;
a plunger driver configured to act on the syringe plunger to move the syringe plunger through the housing during a needle insertion phase and an injection phase;
a safety shroud; and
a plunger driver latch coupled to said safety shroud and moveable between first and second positions and configured to:
while in the first position before the needle insertion phase, latch the plunger driver at a pre-insertion position in the housing,
move to the second position as a result of pressure applied to the safety shroud, to unlatch the plunger driver from the pre-insertion position and allow forward movement of the plunger driver to a post-insertion and injection position in the housing and latch the plunger driver at the post-insertion and injection position, and
return to the first position upon removal or reduction of pressure on the safety shroud, to unlatch the plunger driver from the post-insertion and injection position and allow forward movement of the plunger driver to deploy the safety shroud, wherein the auto-injector comprises a biasing member configured to bias the plunger driver latch towards the first position.

2. The auto-injector according to claim 1, wherein before deployment of the safety shroud, the safety shroud is moveable between an extended position and a retracted position in the housing as a result of said applying, or removal or reduction of pressure.

3. The auto-injector according to claim 2, wherein before deployment of the safety shroud, movement of the safety shroud from the extended position to the retracted position moves the plunger driver latch from the first position to the second position to unlatch the plunger driver from the pre-insertion position.

4. The auto-injector according to claim 3, wherein movement of the safety shroud from the retracted position to the extended position returns the plunger driver latch to the first position to unlatch the plunger driver from the post-insertion and injection position.

5. The auto-injector according to claim 4, wherein the safety shroud comprises a first angled cam surface configured to engage a second angled cam surface of the plunger driver latch to move the plunger driver latch to the second position when the safety shroud is moved to the retracted position.

6. The auto-injector according to claim 5,
wherein the plunger driver latch comprises a pivot; and
wherein the plunger driver latch is rotatable about the pivot to move between the first position and the second position.

7. The auto-injector according to claim 6, wherein the plunger driver latch is configured to:
rotate about the pivot from the first position to the second position responsive to said engagement between the first angled cam surface and the second angled cam surface, and
rotate about the pivot from the second position to the first position responsive to disengagement of the first angled cam surface from the second angled cam surface.

8. (canceled)

9. The auto-injector according to claim 1,
wherein the biasing member comprises a spring.

10. The auto-injector according to claim 1,
wherein the plunger driver comprises an engagement member;
wherein the plunger driver latch comprises a first blocking surface and a second blocking surface;
wherein when the plunger driver latch is in the first position, the first blocking surface of is configured to engage the engagement member of the plunger driver to latch the plunger driver at the pre-insertion position; and
wherein when the plunger driver latch is in the second position, the second blocking surface is configured to engage the engagement member of the plunger driver to latch the plunger driver at the post-insertion and injection position.

11. The auto-injector according to claim 1, wherein the plunger driver is configured to decouple from the plunger of the syringe at the post-insertion and injection position.

12. The auto-injector according to claim 1, wherein after said unlatching of the plunger driver from the post-insertion and injection position, the plunger driver is configured apply a continuous forward force to the safety shroud to deploy the safety shroud.

13. The auto-injector according to claim 12, wherein the plunger driver is configured to lock out the safety shroud on deployment thereof.

14. The auto-injector according to claim 1, wherein the auto-injector comprises an openable lid to allow insertion and removal of the syringe, wherein the lid is configured to hinge between an open position and a closed position.

15. The auto-injector according to claim 14, wherein the lid is configured on a closing movement thereof to prime a drive spring of the plunger driver.

16. The auto-injector according to claim 1 in combination with a syringe comprising a syringe safety shroud configured to cover a needle of the syringe when in a closed position.

17. The auto-injector according to claim 16, wherein after said unlatching of the plunger driver from the post-insertion and injection position, the plunger driver is configured apply a continuous forward force to the syringe safety shroud to deploy the syringe safety shroud.

18. The auto-injector according to claim 17, wherein the plunger driver is configured to lock out the syringe safety shroud on deployment thereof.

19. A kit of parts comprising:
the auto-injector of claim 1; and
a syringe comprising a syringe safety shroud configured to cover a needle of the syringe when in a deployed position.

20. The kit of parts according to claim 19, wherein after said unlatching of the plunger driver from the post-insertion and injection position, the plunger driver is configured apply a continuous forward force to the syringe safety shroud to deploy the syringe safety shroud.

21. The kit of parts according to claim 20, wherein the plunger driver is configured to lock out the syringe safety shroud on deployment thereof.
